(19) United States
(12) Patent Application Publication
(10) Pub. No.: US 2006/0025690 A1
(43) Pub. Date: Feb. 2, 2006

(54) Acoustic body examination

(76) Inventors: Jacques Y. Guigne, Paradise (CA); James Stacey, St. John's (CA)

Correspondence Address: LEON D. ROSEN;FREILICH, HORNBAKER & ROSEN, Suite 1220, 10960 Wilshire Blvd., Los Angeles, CA, 90024, US

(21) Appl. No.: 10/901,248

(22) Filed: Jul. 27, 2004

(51) Int. Cl. A61B 8/06

(52) U.S. Cl. 600/459; 600/447

(57) ABSTRACT

Apparatus for making an acoustic analysis of a patient's body includes a glove device (12) with finger receivers (21-25) that receive the fingers of a caregiver, and sensors (51-53) that indicate the relative positions and orientations of the finger receivers. An acoustic transducer device (31-35) is mounted on each finger receiver, and possibly also (42) on the palm region (44) and other areas of the glove device. While transducer devices are pressed against the patient's body (62), electronic circuitry (112) generates electrical pulses that drive the transducers to transmit acoustic beams into the patient's body. A data processor (140) processes outputs from the transducers that represent acoustic echoes or transmitted beams received from regions of different densities in the patient's body. By mounting the acoustic transducer devices on finger receivers, the caregiver can rapidly and easily position the acoustic transducer devices at selected positions on the patient's body.

### BACKGROUND OF THE INVENTION

Conventional ultrasound transducers for noninvasive probing of a patient's body, often include a fixed array of transducers connected to a flexible cable. The cable extends to circuitry that generates electrical pulses that cause the transducers to produce acoustic pulses. The circuitry also includes a data processor for processing data representing echoes of the acoustic pulses. The echoes can be detected by the same transducers that produce the acoustic pulses, or by separate transducers that are used only for detection. The circuitry that generates electrical pulses controls the output of the array of transducers so the acoustic beam has a narrow spread angle, and so the acoustic beam can be steered so it passes in a desired direction from the array.

The array can be pressed against a selected location on the patient's body, with acoustic gel used for efficient acoustic coupling, as to detect a region of large change in density. The acoustic echoes of the transmitted beam can be used to generate a display indicating change in density at a certain depth within the pressed-against body location. However, there are typically multiple changes in density between the skin and the location of interest, which create multiple echoes that appear as speckle, clutter and other noise artifacts and that reduce contrast at strongly interacting tissue interfaces. An acoustic probe device that could be easily used by a caregiver to produce echoes that the data processor could use to produce a display or other indication of tissue density, with reduced noise artifacts, would be of value.

### SUMMARY OF THE INVENTION

In accordance with one embodiment of the present invention, an acoustic body probe is provided, which facilitates the making of an acoustic analysis of a location on a patient's body, and which enables the use of a plurality of separated acoustic transducers together to obtain a better analysis. The acoustic body probe includes a glove device that can be worn on a person's hand and that includes at least one and preferably a plurality of finger receivers that receive fingers of the caregiver. The acoustic body probe also includes a plurality of separate sonic transmit/receive transducer devices, each mounted on a different one of the finger receivers and/or elsewhere, that can be each pressed against a different location on a region of the patient's body simply by the caregiver's fingers pressing the transducers against such different locations. The outputs of a plurality of different transducer devices can be numerically processed to reduce artifacts (spurious signals) that would be produced by any one of them alone. The outputs are used to produce one or more images for human analysis.

The glove device is of the type that produces signals indicating the relative positions of the different finger receivers, and of the different acoustic transducers that are used together. This enables a more rigorous analysis of the patient by taking into consideration the positions of the transducers.

The glove device includes a palm area that covers the palm of the caregiver. An array of acoustic transducers can be mounted on the palm area to provide an additional location where acoustic energy can be detected and/or transmitted. A large palm area allows a larger array of acoustic transducers to be located there than on the finger receivers.

Pressure sensors can lie between each transducer device and the glove device. Body tissue is malleable, and measurement of the force with which each transducer device presses against the body can be useful in evaluating the acoustic output signals.

The novel features of the invention are set forth with particularity in the appended claims. The invention will be best understood from the following description when read in conjunction with the accompanying drawings.

### BRIEF DESCRIPTION OF THE DRAWINGS

FIG. 1 is an isometric view of a glove device of the present invention.

FIG. 2 is a sectional view of two acoustic transducer devices on two finger receivers of the glove device of FIG. 1, shown being pressed against different locations on a patient's body by a caregiver.

FIG. 3 is a sectional view of two acoustic transducer devices, one on a finger receiver and the other on a palm area of the glove device of FIG. 1.

FIG. 4 is a schematic diagram of an acoustic analysis system which includes circuitry that generates electrical pulses for driving acoustic transistors of the glove device of FIG. 1, and that includes a data processor for processing the outputs of acoustic transducers that detect echoes of acoustic energy pulses.

FIG. 5 is a front elevation view of one of the transducer devices of FIG. 2.

FIG. 6 is a sectional view showing transducers on four fingers and the palm being used together.

### DESCRIPTION OF THE PREFERRED EMBODIMENTS

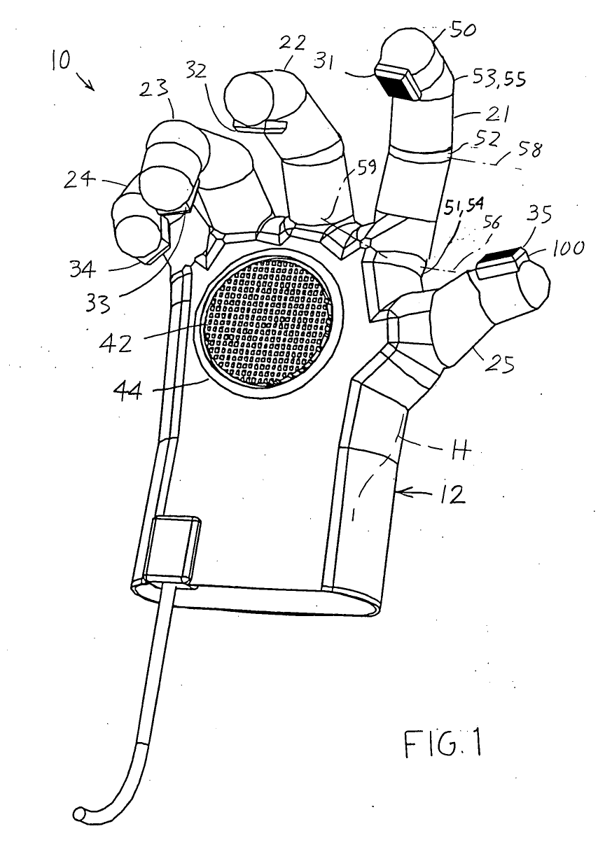

FIG. 1 illustrates apparatus 10 for making an acoustic analysis of a patient, which includes a glove device 12 for receiving the hand H of a physician, technician, or other caregiver. The glove device includes five finger receivers 21-25 that each receives one of the fingers of the caregiver's hand, the particular glove device being constructed for mounting on the right hand. Five separate acoustic transducer devices 31-35 are mounted on the finger receivers.

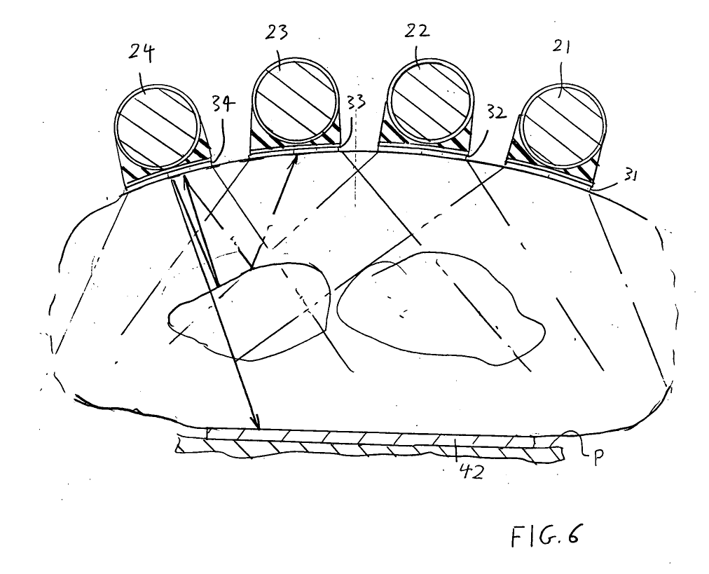

Each acoustic transducer device 31-35 preferably includes an array of sonic transducers, preferably a two-dimensional array of at least 3×3=9 transducers, such as the 8×8 array 31 in FIG. 5 of sixty-four transducers 40. The array allows pulses delivered to the sixty-four transducers to be phased so as to produce a narrow beam (a beam with a narrow spread angle). The beam spread angle can be reduced by using an array with more transducers. The narrow spread angle increases the resolution of the resulting images. The number of transducers in the array should be the maximum number that can be accommodated in the limited available area, subject to cost restraints. An additional transducer device 42 (FIG. 1) which comprises a larger array of transducers, lies at a palm portion 44 of the glove device.

The glove device is of a type that enables detection of the positions and orientations of different locations on the glove, such as the fingertips 50 where the transducer devices 31-35 are located. The relative positions of the fingertips can be determined by position sensors indicated at 51-53 that detect pivoting of different joints of each finger. Examples of such glove devices is a “Cyberglove” sold by VR Glove Technologies Immersion Corp., and a “5DT Data Glove” sold by the 5DT company. Bending of the finger receivers, especially at joints at 54 and 55 and especially about lateral axes 56 and 58, enables selection of the position and orientation of the transducer devices 31-35 in the fingers above the joint at 52. The finger receivers preferably also allow pivoting about a longitudinal axis 59.

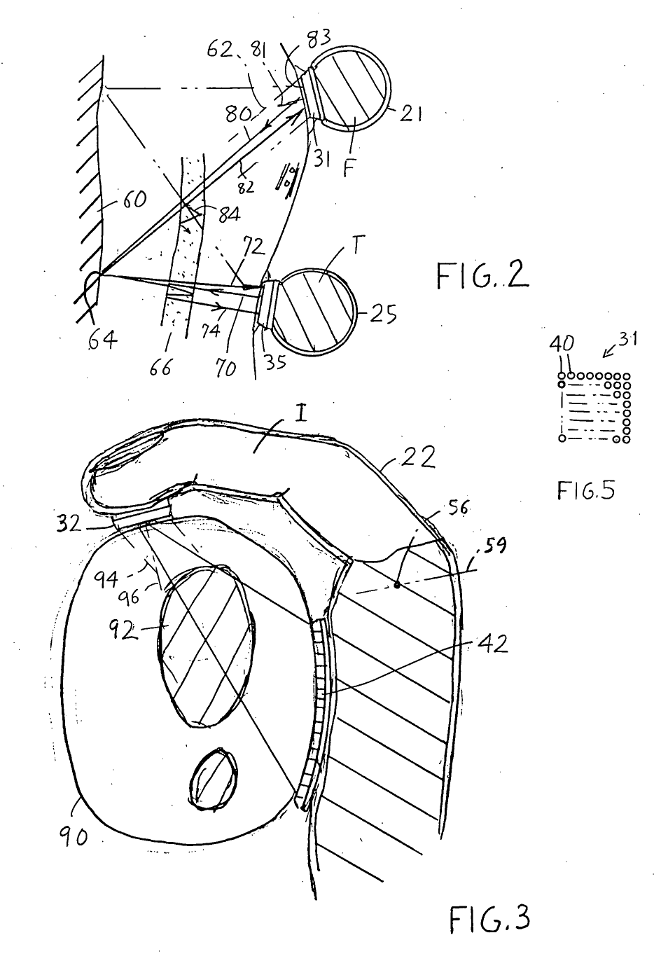

FIG. 2 illustrates a situation where an area of interest 60 of a patient's body 62 is probed by two transducer devices 35, 31 on finger receivers located respectively on the forefinger F and thumb T of the caregiver. Each transducer can produce a narrow acoustic beam such as 63, which can be steered to pass in a desired direction, by controlling the relative phases of pulses applied to individual transducers of the array. The first acoustic transducer device 35 produces a pulsed acoustic beam moving along path 70 and detects echoes from a location 64 traveling along path 72. An echo from location 64 traveling along path 72 will arrive back at the transducer device 35 at a predetermined instant after the original acoustic pulse 70 was generated. However, internal reflections at a region 66 will result in an additional acoustic echo 74 arriving at the same time, which tends to mask the desired echo at path 72.

The second transducer device 31 also probes the location of interest 64, when transducer device 35 is not operating. The location of device 31 relative to the first device is known by the position sensors in the glove device. The direction of the beam and time delay at which an echo is detected are adjusted accordingly. Device 31 produces a pulsed beam 80 that is reflected from location 64 to produce an echo 82. The beam is steered to move at an angle of a plurality of degrees to the direction in which the transducer device face 83 is facing. A portion of the original beam 80 is reflected at the region 66 to produce reflections 84, but the reflections 84 are not received back at the transducer 31 at the time that the echo 82 is received. Thus, the region 66 produces an artifact in the output of transducer device 35 but not in transducer device 31. This difference can be used to reduce artifacts that degrade the signal. In actuality, there will be many internal reflections of the original pulse from blood vessels, etc. The use of two or more transducer devices that probe the location of interest from different locations enables the generation of a clearer image of the location of interest.

FIG. 3 illustrates a situation in which a transducer device 32 and the large transducer array 42 are placed on sides of a portion 90 of the patient's body, that are angled about 120°, but not close (within 100) of exact opposite sides. This setup is used to analyze a region 92 of the body. An acoustic energy beam 94 is directed through the region 92 and against the large transducer array 42. Here, the device 32 is used solely to transmit a beam and the large array 42 is used solely as a sonic detector. The beam is steered, in that its direction is controlled to be at an angle of plurality of degrees from a line 96 that is perpendicular to the direction in which the transducer array faces. Such steering allows selection of the direction of transmissibility of the beam to scan different parts of region 92 without moving the transducer device. The two transducer devices 32, 42 are not located at precisely opposite locations of the body, but their relative positions are known by the position sensors of the glove device.

The glove device 12 of FIG. 1 is a convenient device for holding one or a plurality of transducer devices and for pressing a selected one or more against selected locations on the human body. It would be possible to position each transducer device on a moveable platform such as a piston, and move and tilt each device against a selected location on the body, and to detect the position of each transducer device. However, by mounting the transducer devices on a glove that uses the human hand to move and position each transducer device against a location and in a selected orientation selected by the person wearing the glove, the positioning is accomplished much more rapidly and with a much smaller apparatus. Since high technology gloves are readily available, which detect the position and orientation of different parts of the human hand, especially the fingers, the major modification to such gloves is to mount the transducer devices on them and connect electrical conductors to the transducer devices.

When applicant's transducer devices are pressed against the body, body tissue deforms, with the deformation dependent upon the force that is applied. The level of applied force can help in constructing a truer image of body features. Such forces are readily determinable by placing compression force sensors 100 (FIG. 1) lying between each transducer device and a corresponding location on the glove device.

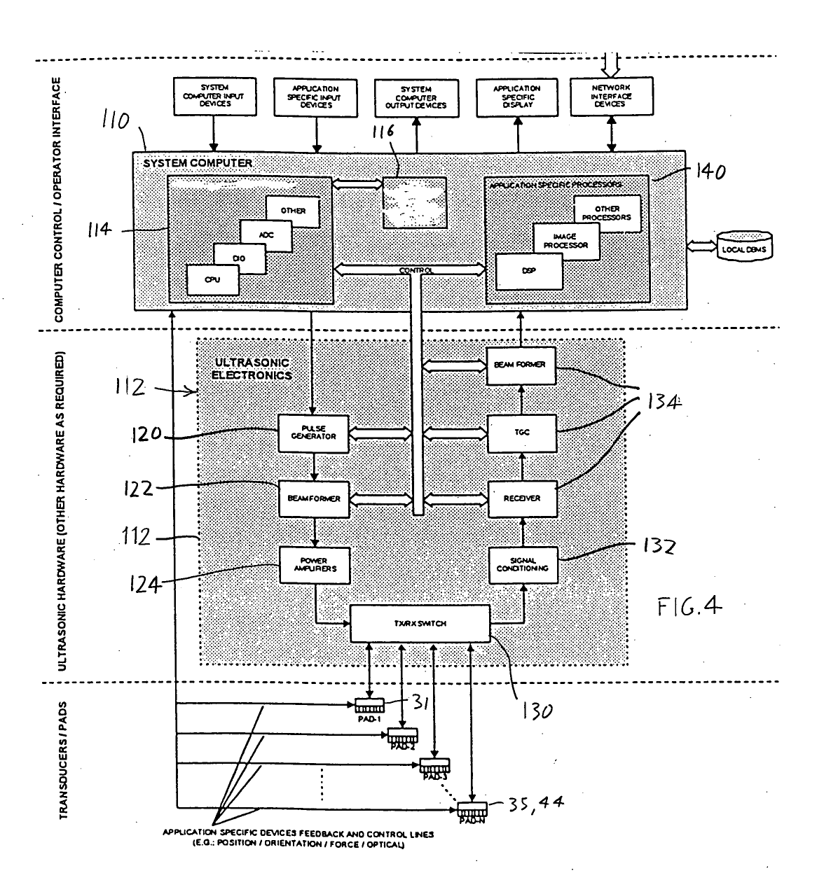

FIG. 4 is a schematic diagram of a system for driving and analyzing the transducer devices. The system includes a system computer 110 with software that controls electronic circuitry 112 that drives the transducers such as 31-35 and 44 to produce sonic beams within the human body and to detect the transmitted sonic beams or their echoes. The electronics include a pulse generator 120, a beam former 122 that controls the phases of pulses to produce narrow (or wide) beam spread angles and to determine the direction of propagation of the beam, and an amplifier 124. The output of the amplifier is directed through a switch 130 to the transducer devices to energize them. The computer 110 includes a driver 114 that receives signals from position sensors 116 on the glove device, and that controls the electronic circuitry 112 partially in accordance with the positions and orientations of the transducer devices.

When the transducer devices are not energized, the switch 130 delivers outputs from the transducer devices that represent echoes or detected direct transmission beams, to a signal conditioning circuit 132. The output from the signal conditioning circuit 132 is delivered through additional processing circuitry 134 to the system computer 110. In most cases, an application-specific processor 140 of the system computer displays data representing detected sonic energy as an image on a screen. Where at least two transducers are used to each detect sonic energy, especially echoes, the outputs of the two transducer devices can be used to construct a three-dimensional image. Perhaps the most practical display is a display of echoes received from a plurality of transducer devices, with speckles, clutter and noise reduced by eliminating noise detected by one transducer device but not by others at the same location in the body.

FIG. 6 shows transducers on four fingers 21-24 and a transducer 42 on the palm P being used to probe a region of the patent's body. The transducers 31-34 on the fingers generate beams with a large spread angle (e.g. 80°) so they produce overlapping views of the same locations in the body. The finger transducers can be used to detect reflections, while the palm transducer is used to detect transmissions through the body region.

Thus, the invention provides apparatus for sonically probing a body, which facilitates positioning of sonic transducer devices against selected locations on the body. This is accomplished by mounting one or more transducer devices on a flexible holder that not only facilitates movement and tilting of the transducer devices, but which can provide an output indicating the positions and orientations of the transducer devices. Transducer devices are preferably mounted on finger receivers of a glove device, and especially of a glove device that produces an output indicating the positions and orientations of the finger receivers. A larger array of transducers can be mounted on the palm area of the glove device.

Although particular embodiments of the invention have been described and illustrated herein, it is recognized that modifications and variations may readily occur to those skilled in the art, and consequently, it is intended that the claims be interpreted to cover such modifications and equivalents.

What is claimed is:

1. Apparatus for making an acoustic analysis of a part of a patient's body, which includes electronic circuitry comprising a circuit that generates electrical pulses for driving acoustic transducers used as acoustic transmitters and a data processor for processing data representing the output of acoustic transducers used as acoustic detectors, comprising:
a plurality of acoustic transducer devices with at least one that generates an acoustic beam and at least one that detects acoustic energy;
a glove device that has a plurality of bendable finger receivers, at least a first of said acoustic transducer devices being mounted on a first of said finger receivers so a person can easily move the first transducer device against a selected location on the patient's body;
a plurality of position sensors mounted on said glove device for indicating at least the relative positions of said transducer devices.

2. The apparatus described in claim 1 wherein:
said plurality of acoustic transducer devices each includes an array of transducers for generating a directed acoustic beam, whereby to direct a beam in a desired direction from the location of each transducer device without having to tilt the transducer device.

3. The apparatus described in claim 1 wherein:
said glove device has a palm portion that lies over the palm of the wearer's hand;
one of said acoustic transducer devices is mounted on said palm portion of said glove device.

4. The apparatus described in claim 1 wherein:
said plurality of finger receivers includes a forefinger receiver that allows bending about lower and middle joints of a forefinger lying therein.

5. The apparatus described in claim 1 including:
a pressure sensor lying between said first finger receiver and said first acoustic transducer device, for sensing the force of the first transducer device against the patient's body.

6. Apparatus for making an acoustic analysis of a patient, which includes electronic circuitry that includes a circuit that generates electrical pulses for driving acoustic transducer devices and a data processor for processing data representing the output of an acoustic detector, comprising:
a plurality of acoustic transducer devices that each can generate acoustic energy, and at least one transducer that is useful as an acoustic detector that can detect acoustic energy, said transducers and at least one detector being connected to said electronic circuitry;
a glove device with a plurality of finger receivers that can be bent with respect to one another, each of a plurality of said acoustic transducers is mounted on a different location on said glove device, with at least one transducer device mounted on one of said finger receivers, so a plurality of said acoustic transducer devices all can be simultaneously pressed against a curved surface of the patient's body.

7. The apparatus described in claim 6 wherein:
each of said acoustic transducer devices comprises a two-dimensional array of acoustic transducers whereby to form a directed beam.

8. The apparatus described in claim 6 including:
means in said flexible holder which generates position indicating signals that indicate at least the position of each of said transducer devices relative to each other, said means for generating position indicating signals being connected to said data processor.

9. The apparatus described in claim 6 including:
a plurality of pressure sensors that each lies between one of said finger receiver and a corresponding one of said transducer device.

10. A method for a caregiver to sonically probe a part of a patient's body, comprising:
wearing a glove device on the caregiver's hand and pressing a finger of the caregiver's hand toward a first location on the patient's body to press a first acoustic transducer device on a finger receiver against said location on the patient's body;
while pressing said first acoustic transducer device against the patient's body, transmitting electrical pulses to said acoustic transducer device to cause said acoustic transducer device to transmit sonic energy into the patient's body to a body interior location, while detecting echos of the sonic energy and delivering electrical signals representing the echos to a signal processing circuit.

11. The method described in claim 10 including:
pressing a second finger of the caregiver's hand toward a second location on the patient's body to press a second acoustic transducer device against the second location and transmitting second sonic energy from said second location to said body interior location, while detecting echos of said second sonic energy.

12. The method described in claim 10 including:
forming sonic energy from said first and second transducer devices as directed beams which are non parallel to each other to interrogate a same region of the patient's body part.

13. A method for a caregiver to sonically probe a part of a patient's body, comprising:
holding a glove device in the caregiver's hand and using a finger of the caregiver's hand to press a first finger receiver of the glove device toward a first location on the patient's body to press a first acoustic transducer device on the first finger receiver against said location on the patient's body, while simultaneously pressing a second acoustic transducer device against a second location on the patient's body that is angled from said first location, but that is angled by no more than 170° from said first location, so a sonic beam is not directed perpendicular to one of said transducer devices towards the other one, each of said transducer devices having a face;
energizing one of said transducer devices to produce a sonic beam that is steered to extend at an angle to a line perpendicular to a face of said one of said transducer devices toward said first location on the patient's body.
